(19) United States
(12) Patent Application Publication
(10) Pub. No.: US 2007/0123903 A1
(43) Pub. Date: May 31, 2007

(54) Medical Device installation tool and methods of use

(75) Inventors: Douglas Raymond, Randolph, MA (US); Craig Hoyle, Woonsocket, RI (US); Shinikequa White, Dorchester, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: DePuy Spine, Inc., Raynham, MA (US)

(21) Appl. No.: 11/263,393

(22) Filed: Oct. 31, 2005

(51) Int. Cl. A61F 2/00

(52) U.S. Cl. 606/99

(57) ABSTRACT

Methods and devices for implanting a prosthetic device, such as an artificial spinal implant, are provided. The installation tool can include a handle having a pair of opposed levers, an optional pusher block disposed between the levers, and a shaft at least partially disposed within the handle and able to be coupled to the pusher block and/or to a prosthetic device. As the shaft translates along a longitudinal axis of the installation tool, the pusher block and/or the prosthetic device separate the levers and distract adjacent vertebral bodies to position a prosthetic device therebetween. The tool is able to maintain its an overall length during use, and it can be configured in rotation and/or translation modes.

### FIELD OF THE INVENTION

The invention relates broadly to a tool for inserting a prosthesis within a body, and more particularly to a tool for inserting prostheses, such as artificial discs or other implants within an intervertebral space.

### BACKGROUND OF THE INVENTION

Spinal surgery involves many challenges as the long-term health and mobility of the patient often depends on the surgeon's technique and precision. One type of spinal surgery involves the removal of the natural disc tissue that is located between adjacent vertebral bodies. Procedures are known in which the natural, damaged disc tissue is replaced with an interbody cage or fusion device, or with a disc prosthesis.

The insertion of an article, such as an artificial disc prosthesis, presents the surgeon with several challenges. The adjacent vertebral bodies collapse upon each other once the natural disc tissue is removed. These bodies must be separated to an extent sufficient to enable the placement of the prosthesis. However, if the vertebral bodies are separated, or distracted, to beyond a certain degree, further injury can occur. The disc prosthesis must also be properly positioned between the adjacent vertebral bodies. Over-insertion or under-insertion of the prosthesis can lead to pain, postural problems and/or limited mobility or freedom of movement.

Specialized tools have been developed to facilitate the placement of devices, such as disc prostheses, between adjacent vertebral bodies of a patient's spine. Among the known tools for performing such procedures are separate spinal distractors and insertion devices. The use of separate tools to distract the vertebral bodies and insert a disc prosthesis or graft can prove cumbersome. Further, the use of some distractors can cause over-distraction of the vertebral bodies.

Despite existing tools and technologies, there remains a need to provide a device to facilitate the proper and convenient insertion of an object, such as a disc prosthesis, between adjacent vertebral bodies while minimizing the risk of further injury to the patient.

### SUMMARY OF THE INVENTION

The present invention generally provides methods and devices for facilitating the proper and convenient insertion of an object, such as a disc prosthesis, between adjacent vertebral bodies. In one embodiment, a medical device installation tool can include a housing, a pair of opposed levers, and a prosthesis positioning mechanism at least a portion of which is disposed between the pair of opposed levers. The opposed levers can each have a proximal end and a distal end, the proximal end of each lever being moveably coupled to a portion of the housing. The prosthesis positioning mechanism can be selectively configured such that at least a portion of the prosthesis positioning mechanism translates along a longitudinal axis of the installation tool while maintaining a substantially fixed length of the installation tool.

In yet another embodiment, a medical device installation tool can include a housing, a shaft coupled to the housing and a pair of opposed levers, each having a proximal end and a distal end wherein the proximal end of each lever can be pivotably coupled to a portion of the housing such that the distal ends are configured to separate in response to the movement of one or more objects between the levers in the proximal to distal direction. The tool can be selectively configured such that the shaft will translate along a longitudinal axis of the installation tool or will rotate about the longitudinal axis of the installation tool as a result of manipulation of a single driver. For example, the medical device installation tool can include an actuator that can be configured in a first position that allows the driver to effect translation of the shaft along the longitudinal axis of the installation tool, and a second position that allows the driver to effect rotation of the shaft about the longitudinal axis of the installation tool.

Methods for implanting a prosthetic device are also provided. In one embodiment, the method can include disposing portions of opposed, pivotable levers of an installation tool between vertebral bodies. The method can further include linearly translating a shaft along a longitudinal axis of the installation tool to move a pusher block and/or a prosthetic device between the opposed levers toward the vertebral bodies while causing distal ends of the opposed levers to separate and distract the vertebral bodies to implant the prosthetic device between the distracted vertebral bodies while maintaining the overall length of the tool. When the implant reaches its final position, continued translation of the shaft draws the opposed levers from the disc space leaving only the implant in the disc space. If the shaft is connected directly to a prosthesis, the method can further include rotating the shaft about its longitudinal axis to decouple the installation tool from the prosthetic device and linearly translating the shaft along the longitudinal axis of the installation tool to cause the levers to retract from the vertebral bodies.

### BRIEF DESCRIPTION OF THE DRAWINGS

The invention will be more fully understood from the following detailed description taken in conjunction with the accompanying drawings, in which:

FIG. 1 is a perspective view of one embodiment of an installation tool;

FIG. 1A is a perspective view of another embodiment of an installation tool;

FIG. 2 is an assembly view of the installation tool of FIG. 1;

FIG. 3 is a cross-sectional view of a prosthesis positioning mechanism according to one embodiment of installation tool showing;

FIG. 4 illustrates a sectional view of a shaft and housing of the installation tool of FIG. 3 taken along section 4-4;

FIG. 5 illustrates an embodiment of an installation tool that provides linear translation and rotational motion of a shaft of the tool;

FIG. 6 illustrates a sectional view of an interface between an actuator and the shaft of the installation tool of FIG. 5 taken along section 6-6;

FIG. 7 illustrates another embodiment of an installation tool that provides linear translation and rotational motion of a shaft of the tool;

FIG. 7A illustrates a sectional view of an interface between an actuator and the shaft of the installation tool of FIG. 7 taken along section 7A-7A;

FIG. 8 illustrates an embodiment of the installation tool in use during an initial stage of inserting a prosthesis between adjacent vertebrae;

FIG. 9 illustrates the installation tool of FIG. 8 in use to insert a prosthesis between adjacent vertebrae, distracting the adjacent vertebrae;

FIG. 10 illustrates the installation tool of FIG. 8 during a further stage of inserting a prosthetic device between the adjacent vertebrae;

FIG. 11 illustrates decoupling a shaft of the installation tool of FIG. 8 from the prosthetic device after inserting a prosthesis between adjacent vertebrae; and

FIG. 12 illustrates the installation tool of FIG. 8 being withdrawn from between the adjacent vertebrae.

### DETAILED DESCRIPTION OF THE INVENTION

Certain exemplary embodiments will now be described to provide an overall understanding of the principles, structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with features of other embodiments. Such modifications and variations are intended to be included within the scope of the present invention.

The present invention provides a medical device installation tool for implanting a prosthetic device, such as a spinal implant, between adjacent vertebral bodies. In general, the installation tool includes a proximal housing from which a pair of opposed levers extend distally. The installation tool also includes a shaft that is at least partially disposed within the housing and a movable handle, which is or forms part of a driver, connected to the shaft. In one aspect a pusher block is coupled to or able to be coupled to a distal end of the shaft. The pusher block is, in turn, adapted to be disposed between the levers, and distal movement of the pusher block between the levers causes separation of the levers by the pusher block and/or the prosthesis acting on the levers. Alternatively, the distal end of the shaft is attached directly to a prosthesis, which is adapted to be positioned between the levers, and distal movement of the prosthesis between the levers causes separation of the levers. The installation tool can be configured such that movement (e.g., rotational movement) of the handle causes either rotation of the shaft about its longitudinal axis or translation of the shaft along the longitudinal axis of the installation tool. Among the advantages of the installation tool is that the overall length of the device does not change during use, regardless of whether the tool is used in the shaft rotation of shaft translation modes.

The installation tool can be provided as a kit having modular components which allow the surgeon to select from among a variety of components to assemble an installation tool that is optimized for its intended use. Although the invention is described primarily with reference to use of the tool to install an artificial disc between adjacent vertebral bodies, it is understood that the installation tool of the invention can be used to place other elements between vertebral bodies, or in other locations within a patient's body. Exemplary elements that can be placed between vertebral bodies include, but are not limited to interbody cages, fusion devices, spacers, grafts, and the like.

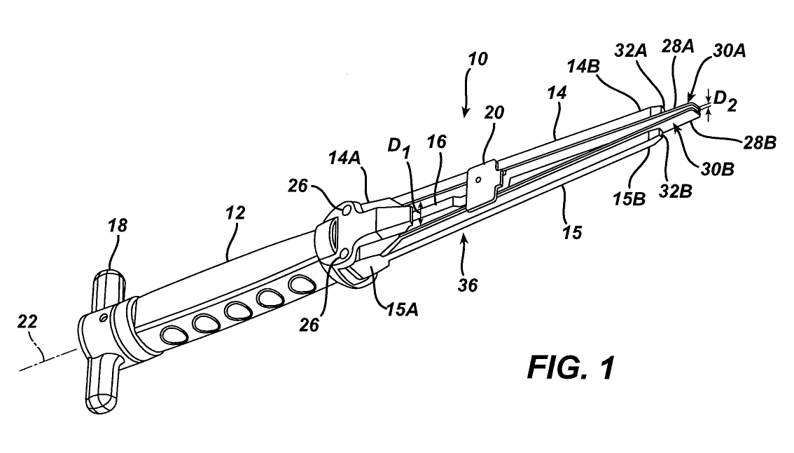

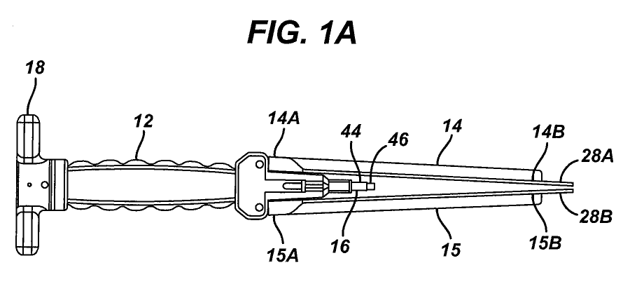

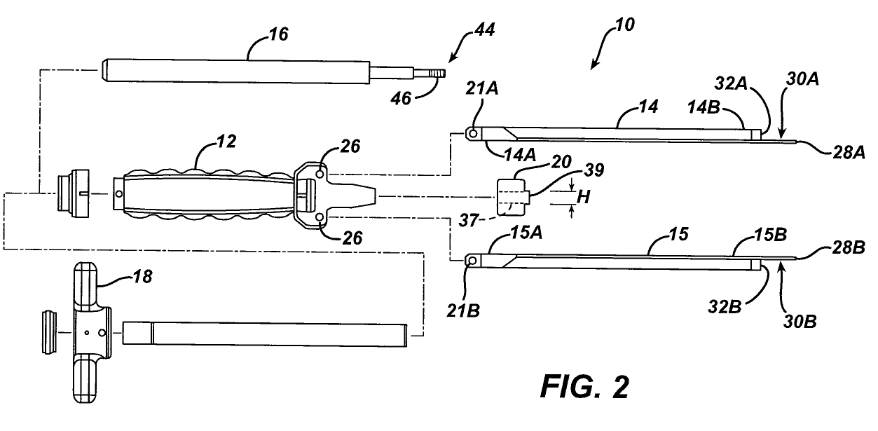

FIGS. 1-2 illustrate one embodiment of an installation tool 10 having a housing 12 which can facilitate grasping and manipulation of the tool 10, and a pair of opposed levers 14, 15 that extend distally from the housing 12. The installation tool 10 also includes a movable (e.g., rotatable) handle 18 at a proximal end of the housing 12 and a shaft 16, coupled to the handle 18 by way of a drive shaft 64 and at least partially disposed within the housing 12. In one embodiment, shown in FIGS. 1 and 2, a distal end 44 of the shaft 16 extends from the housing 12 and is coupled to a pusher block 20. As discussed below, the pusher block 20 can be attached to or disposed adjacent to an implant during use of the installation tool 10. In another embodiment, shown in FIG. 1A, the distal end 44 of shaft 16 is adapted to connect directly to an implant 100 without an intervening pusher block. One skilled in the art will appreciate that the installation tool 10 can be provided as modular kit that will enable a user to attach or remove the pusher block, or to use pusher blocks of different shapes and sizes, as required by a given application.

The opposed first and second levers 14, 15, each have a proximal end 14A, 15A and a distal end 14B, 15B, respectively. The proximal ends 14A, 15A of each lever 14, 15 can be pivotably coupled to the housing 12 of the installation tool 10 to allow each of the levers 14, 15 to pivot about its attachment point. For example, the proximal end 14A of the first lever 14 and the proximal end 15A of the second lever 15 can each include a bore 21A, 21B, which seats pivot pins 26 to pivotally mount each lever to the housing. As the levers 14, 15 pivot about pins 26, the distal ends 14B, 15B of the levers 14, 15 separate to facilitate distraction or separation of adjacent vertebral bodies as explained below. One skilled in the art will appreciate that the coupling of the levers 14, 15 to the housing 12 can be done in such a way as to allow some play (e.g., linear movement) to facilitate convenient use and to accommodate anatomical features or irregularities. For example, the levers 14, 15 can each include a slot which seats about the pivot pins 26 to allow some linear translation of the levers 14, 15 relative to the housing 12. One skilled in the art will also appreciate that the levers 14, 15 can be detachably coupled to the housing 12 to allow attachment of various types of levers to the housing, such as levers having varying geometries.

The distal ends 14B, 15B of the levers 14, 15 can include blade tips 28A, 28B sized and configured to facilitate their placement between vertebral bodies. The blade tips 28A, 28B include outwardly facing surfaces 30A, 30B that can be beveled or radiused. In one embodiment, outwardly facing surfaces 30A, 30B can be substantially curved or angled in a superior or inferior direction to facilitate placement of the blade tips 28A, 28 between adjacent vertebrae.

The distal ends 14B, 15B of the levers 14, 15 can include stop surfaces 32A, 32B disposed adjacent to the blade tips 28A, 28B. The stop surfaces 32A, 32B can be configured to abut a vertebral body during a surgical procedure for installing a prosthesis, such as an artificial disc, between adjacent vertebral bodies. The stop surfaces 32A, 324B can have a variety of geometric configurations. In one embodiment, the stop surfaces 32A, 32B can have a substantially concave profile when viewed in the vertical plane.

The facing surfaces of levers 14, 15 are adapted and configured to allow a prosthetic device to be positioned and guided therebetween. For example, in one embodiment the facing surfaces of levers 14, 15 can include substantially planar surfaces that can guide and/or support the prosthetic device as it moves distally along the levers 14, 15. In another embodiment, the facing surfaces of levers 14, 15 can be configured to support a portion of a prosthesis positioning mechanism, such as a pusher block 20. For example, the pusher block 20 can be coupled to the facing surfaces of levers 14, 15, or to other portions of the levers 14, 15, to minimize rotational motion of the pusher block 20 about the longitudinal axis 22 of the insertion tool 10.

The shaft 16 serves as part of a prosthesis positioning mechanism, and the tool can be configured so that shaft 16 is capable of rotational movement or translational movement (e.g., to position a prosthetic device between adjacent vertebral bodies) while maintaining a substantially fixed overall length of the installation tool 10. While the shaft 16 can be configured in a variety of ways, in one embodiment it is a generally elongate member such as a rod. One skilled in the art will appreciate that other geometries can be used as well. As illustrated in FIGS. 1-3, a proximal end of the shaft is disposed within the housing 12 and a distal end 44 (FIG. 2) can extend from the housing 12 and be disposed between the levers 14, 15. As noted above, the shaft 16 can be adapted for translational movement along the longitudinal axis 22 of the installation tool 10 to position a prosthetic device between adjacent vertebral bodies. During such translation at least a portion of the shaft 16 remains disposed within the housing 12 and no portion of the shaft 16 extends proximally from the handle 18 or substantially beyond the distal portion of the levers 14, 15. Accordingly, the installation tool 10 substantially maintains its overall length during use of the tool 10.

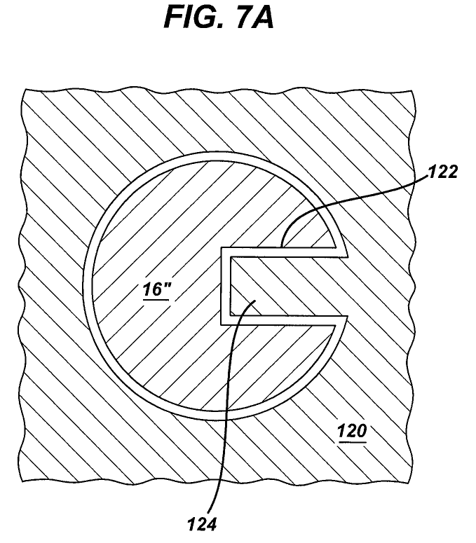

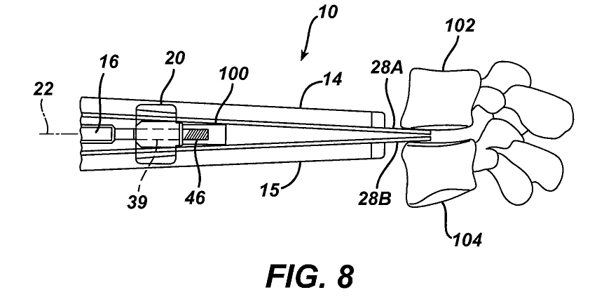

With further reference to FIGS. 1-2, the distal end 44 of the shaft 16 may include a coupling mechanism, such as threaded tip 46, that can be coupled to a prosthetic device 100 (FIG. 8) and/or to pusher block 20. The coupling mechanism 46 can attach to a corresponding coupling mechanism carried by the pusher block 20 and/or a prosthetic device. For example, the prosthetic device or pusher block 20 can include a threaded bore matable with the threaded end 46 of the shaft 16. With such a coupling, forward and rearward motion of the shaft 16 will effect corresponding motion of the distal end of the shaft 16 along longitudinal axis 22 and any prosthesis and/or pusher block 20 attached thereto.

As noted above, the installation tool 10 is designed such that linear translation of a pusher block and/or prosthetic device along the levers 14, 15 in a proximal to distal direction causes the opposed levers 14, 15 to separate. Such separation will enable the levers 14, 15 to distract two adjacent bodies during an installation procedure as discussed below.

In one embodiment, illustrated in FIG. 1, the installation tool 10 includes a pusher block 20 that can also form part of a prosthesis positioning mechanism. The pusher block 20 can be coupled to the distal end 44 of the shaft 16 and disposed between the levers 14, 15. Linear translation of the shaft 16 can cause the pusher block 20 to move between the levers 14, 15 in a proximal to distal direction. As the pusher block 20 (and any attached prosthesis) moves distally, such movement will cause the levers 14, 15 to pivot about their respective pivot pins 26 and separate the distal ends 14B, 15B and blade tips 28A, 28B of the levers 14, 15 from each other. For example, in a closed or at-rest state, the pusher block 20 (and any attached prosthesis) can be positioned in proximity to the proximal ends 14A, 15A of the levers such that the proximal ends 14A, 15A are separated by a distance D₁ and the blade tips 28A, 28B are separated by a distance D₂, where D₂<D₁ as shown in FIG. 1. As the pusher block 20 moves from the proximal end to the distal end of the levers 14, 15, the pusher block 20 (and any attached prosthesis) separates the blade tips 28A, 28B of the installation tool 10, thereby increasing the distance D₂ between the blade tips 28A, 28B.

In one embodiment, the size (e.g., height) of the prosthetic device can determine the amount of separation required between the blade tips 28A, 28B, and thus the amount of distraction required of the vertebral bodies to implant a prosthesis. That is, a relatively larger prosthetic device can require greater amount of separation between the blade tips 28A, 28B and a corresponding amount of distraction of the vertebral bodies. As a result, the pusher block 20 and/or prosthesis can be configured to have various heights (H), depending upon the amount of separation required between the blade tips 28A, 28B. One skilled in the art will appreciate that the adjacent vertebrae should only be distracted by an amount sufficient to insert a prosthesis therebetween. Thus, the pusher block and/or prosthesis should be selected to cause only the minimum amount of distraction necessary to implant a prosthesis. To this end, the tool 10 can be provided with multiple, interchangeable pusher blocks 20 having different sizes and shapes. By way of example, while the pusher block 20 can have a variety of configurations, shapes, and sizes, in one embodiment, the height (H) of the pusher block 20 is in the range of about 8.0 mm to 14.0 mm.

In one embodiment, the pusher block 20 can be configured to guide a prosthetic device through the installation tool 10 into the disc space. For example, as shown in FIG. 2, the pusher block 20 can include a leading face 39 configured to contact a prosthetic device. As the pusher block 20 moves distally between the levers the prosthetic device also moves distally. As a result of such movement, the pusher block 20 and/or the prosthetic device cause the levers 14, 15 to separate as they move distally between the levers 14, 15.

The pusher block 20 can also be configured to allow connection of the distal end 44 of the shaft 16 to the prosthetic device. In one embodiment, illustrated in FIG. 2 the pusher block 20 can include a bore 37 extending therethrough. The shaft 16 can extend through the bore 37 such that the shaft 16 is coupled to the pusher block 20 and such that at least a portion of the coupling mechanism 46 of the shaft 16 extends past face 39 of the pusher block 20. In this embodiment, the coupling mechanism 46 can mate directly to the prosthetic device, or it can mate to a connector element which, in turn, can mate to the prosthetic device.

While the pusher block 20 can be configured to allow connection of the distal end 44 of the shaft 16 to the prosthetic device, the pusher block 20 can have other configurations as well. In one embodiment, the pusher block 20 can include a connection mechanism, such as disposed along the face 39 of the pusher block 20, that enables the pusher block 20 to couple directly to the prosthesis device. By way of non-limiting example, the connection mechanism of the pusher block 20 can include a threaded connection, a dovetail connection, a snap-on connection or a taper lock connection.

In another embodiment, illustrated in FIG. 1A, there is no need for a pusher block 20. Instead, the shaft 16 has a distal portion 44 with a coupling mechanism, such as a threaded tip 46. The distal end of the shaft 16 can thus couple directly to a prosthesis, and the prosthesis causes separation of the levers as it travels distally therebetween.

As indicated above, the prosthesis positioning mechanism can translate along a longitudinal axis 22 of the installation tool 10 while maintaining a substantially fixed length of the installation tool 10. In one embodiment, the installation tool 10 can include a driver mechanism that includes handle 18 configured to effect linear translate the prosthesis positioning mechanism along a longitudinal axis of the installation tool 10 while maintaining the substantially fixed length of the tool 10. For example, the handle 18 and the shaft 16 of the prosthesis positioning mechanism can be configured such that rotation of the handle 18 about the longitudinal axis 22 of the insertion tool 10 adjusts a linear position of the shaft 16 and any attached components.

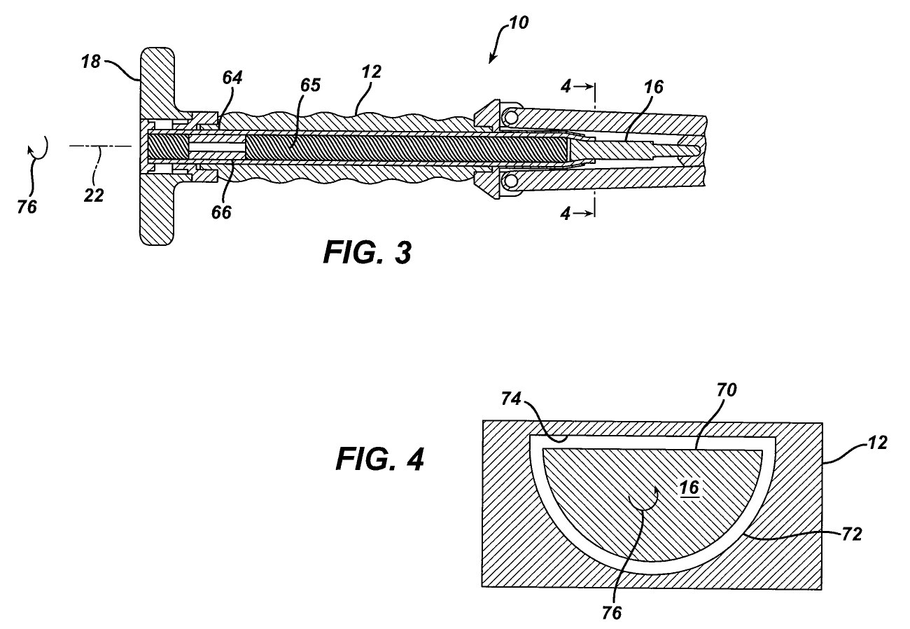

FIG. 3 illustrates one embodiment in which rotation of handle 18 causes only linear translation of the shaft 16. In this embodiment the handle 18 is part of a driver that includes a drive shaft 64. As shown, the handle 18 can be disposed at a proximal end of the housing 12 and it can be configured to receive a rotational force or torque 76. The drive shaft 64 can be disposed within the housing 12 and can be threadably coupled to the proximal end of the shaft 16. In one embodiment, the drive shaft 64 is annular, having internal threads 66 configured to mate with threads 65 disposed about an external surface of the proximal end of the shaft 16.

A portion of the shaft 16 can be rotationally constrained within the housing 12 such that rotation of the threaded drive shaft 64 by the handle 18 can cause linear translation of the shaft 16 along the longitudinal axis 22 of the installation tool 10. For example, a portion of the distal end 44 of the shaft 16 can be “keyed” relative to the housing 12 such that engagement of the housing 12 and the shaft 16 prevents rotation of the shaft 16 when a rotational force is applied to handle 18, thus transferring the rotational force to linear movement of the shaft 16. By way of one example, shown in FIG. 4, the distal end 44 of the shaft 16 can have a cross section with an irregular shape, such as including a flattened surface 70, which fits within a portion of the housing 12 that has a complementary shape, such as a corresponding flattened surface 74. As the threaded drive shaft 64, is rotated, such as by handle 18, constrainment of the shaft 16 by the flattened surface 72 of the shaft 16, prevents rotation of the shaft 16, thereby allowing the shaft 16 to translate along the longitudinal axis 22 of the installation tool 10.

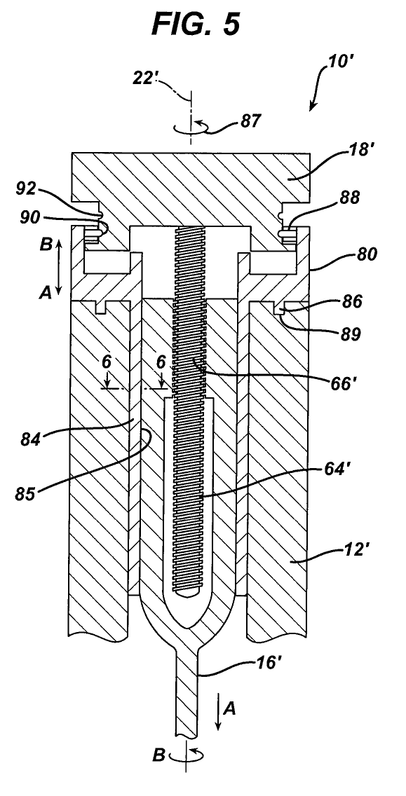

In another embodiment, the installation tool 10 enables a user to select a mode of operation in which rotation of a driver, such as handle 18, causes either linear translation of the shaft 16 or rotation of the shaft 16. Such a design is desirable because linear translation can be useful to implant a prosthesis while rotation of the shaft 16 is useful to couple or decouple the tool 10 and a prosthetic device. FIGS. 5-7A illustrate embodiments of an installation tool that enable both linear translation and rotational movement of the shaft, thereby allowing the tool to both install a prosthetic device and couple to or decouple from a prosthetic device.

One skilled in the art will appreciate that a variety of designs can be implemented to enable the installation tool to be selectively configured to effect linear translation of the shaft 16 or rotation of the shaft 16 upon applying a rotational force to a driver, such as through a handle 18. Generally, a tool with selective linear translation and rotational modes of operation can be provided by rotationally constraining the shaft 16 when a rotational force is applied to a driver, thus enabling the installation tool to operate in a linear translation mode. To effect a rotational mode of operation, the shaft 16 is rotationally unconstrained such that the rotational force applied to a handle 18 effects rotation of the shaft 16.

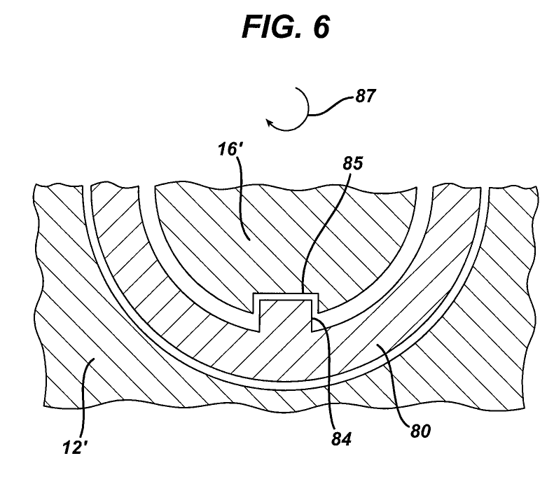

FIGS. 5 and 6 illustrate a portion of one embodiment of an installation tool 10′ that can be selectively configured between linear translation and rotational modes of operation of the shaft 16′. As shown, the installation tool 10′ has a housing 12′, a shaft 16′ disposed within the housing 12′, a handle 18′ threadably coupled to the shaft 16′, and an actuator 80 coupled to the housing 12′. The actuator 80 can be selectively positioned in a first position A that allows linear motion of the shaft 16′ along the longitudinal axis 22′ and a second position B that allows or rotational motion of the shaft 16′ relative to the longitudinal axis 22′.

When the actuator 80 is in position A, the tool is configured for a mode of operation in which the shaft 16′ is rotationally constrained, thereby enabling linear translation of the shaft 16′. As illustrated in FIG. 5, with the actuator 80 in the first position A, the handle coupling portion 88 of the actuator 80 is seated within the first, distal set of detents 90 formed in the handle 18′ and the housing coupling portion 86 of the actuator 80 is mated within the openings 89 formed within the housing 12′. In this configuration the housing coupling portion 86 and the housing 12′ rotationally constrain the shaft 16′ relative to the housing 12′. FIG. 6 illustrates that in the embodiment of FIG. 5, the actuator 80 has a shaft coupling portion 84 that mates within a notch or groove 85 formed in the shaft 16′. As a rotational force 87 is applied to the handle 18′ and drive shaft 64′, interaction between the shaft coupling portion 84 and the notch 85 of the shaft 16′ prevents any rotation of the shaft 16′ and the actuator 80. Thus, the rotational force applied to the handle 18′ will cause the drive shaft 64′ to rotate such that threads 66 of the drive shaft 64′ rotate relative to the threads of the shaft 16′, thereby causing the shaft 16′ to translate along the longitudinal axis 22′ of the installation tool 10′.

With the actuator 80 in the second position B, rotational movement of the shaft 16′ is permitted. The actuator 80 is placed in position B by raising the actuator 80 such that the handle coupling portion 88 of the actuator 80 mates within the second, proximal set of detents 92 formed in the handle 18′, thereby securing the actuator 80 to the handle 18′. At the same time, the housing coupling portion 86 is disengaged from the openings 89 to decouple the actuator 80 and the shaft 16′ from the housing 12′. When a rotational force 87 is applied to the handle 18′, the drive shaft 64′ will rotate, causing both the shaft 16′ and the actuator 80′ to likewise rotate relative to the housing 12′.

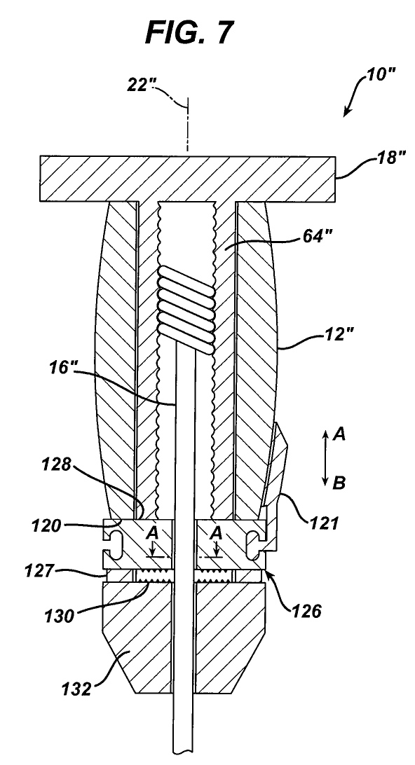

FIGS. 7 and 7A illustrate another embodiment of an installation tool 10″ that can be selectively configured between linear translation and rotational modes of operation of the shaft. As shown, the installation tool 10″ has a housing 12″, a shaft 16″ disposed within the housing 12″, a handle 18″ threadably coupled to the shaft 16″ by way of a drive shaft 64″, and an actuator 120. The actuator 120 is selectively moveable between a first position A that allows rotational motion of the shaft 16″ and a second position B that rotationally constrains the shaft 16″ and allows linear motion of the shaft 16″ along the longitudinal axis 22″. In this embodiment, as shown in FIG. 7A, the actuator can include a shaft coupling portion 124 that mates within a notch or groove 122 within the shaft 16″. Thus, the shaft 16″ and the actuator 120 are coupled together such that one is not able to rotate independent of the other.

The actuator 120 can include a mechanism, such as a switch 121 to control the positioning of the actuator 120 in position A (rotational mode) or position B (linear translation mode). When the actuator 120 is in the first position A, a first, proximal face 128 of the actuator 120 is coupled to the handle 18″, such as by a mechanical coupling or an interference fit between the actuator 120 and a distal portion of the drive shaft 64″. The coupling of the actuator 120 to the shaft 16″ enables rotation of the shaft upon the application of a rotational force to handle 18″. As a rotational force is applied to the handle 18″, the drive shaft 64′ will rotate, causing both the shaft 16″ and the actuator 80′ to rotate.

When the actuator 120 is moved to the second position B, such as by distal movement of the actuator 120, which may result from movement of switch 121, the first, proximal face 128 is detached from its mating connection to the handle 18″. A second, distal face 126 of the actuator 120 is then coupled to a proximal surface 130 on a stationary housing block 132. The coupling of the actuator 120 to the shaft 16″ via the shaft coupling portion 124, as noted above, causes the shaft 16″ to be rotationally constrained. That is, since the actuator 120 and the shaft 16″ are keyed to one another, when the distal face 126 of the actuator 120 is coupled to the stationary housing block 132 any rotation of the handle 18″ and the drive shaft 64″ is not able to cause rotation of the actuator 120 or the shaft 16″. In this configuration, when a rotational force is applied to the handle 18″, the drive shaft 64″ will rotate but the shaft 16″ will not. As a result, the rotational motion of the drive shaft 64″ will be converted to linear motion of the shaft 16″ along the longitudinal axis 22″ of the installation tool 10″.

FIGS. 8-12 sequentially illustrate the use of an installation tool 10 for the implantation of a prosthetic device 100, such as a vertebral disc, between adjacent vertebral bodies 102, 104. As illustrated in FIG. 8, the tool 10 can be assembled in one embodiment with the threaded portion 46 of the shaft 16 extending through the bore 39 of the pusher block 20 and coupled to the prosthetic device 100. For example, the tool can be configured in a shaft rotation mode in which rotation of handle 18 (FIG. 1) will cause the shaft to rotate so that it can be threaded onto prosthetic device 100. In an initial state, the pusher block 20 can be positioned in proximity to a proximal end of the levers 14, 15 such that the blade tips 28A, 28B are in a closed or non-distracted state. The blade tips 28A, 28B can then be inserted or wedged between adjacent vertebral bodies 102, 104 to effect slight separation between the vertebral bodies 102, 104. Although not illustrated, one skilled in the art will appreciate that tool 10 can be manipulated such that the blade tips 30A, 30B are fully inserted between the vertebral bodies such that the stop surfaces 32A, 32B of the levers 14, 15 can abut a surface of the vertebral bodies.

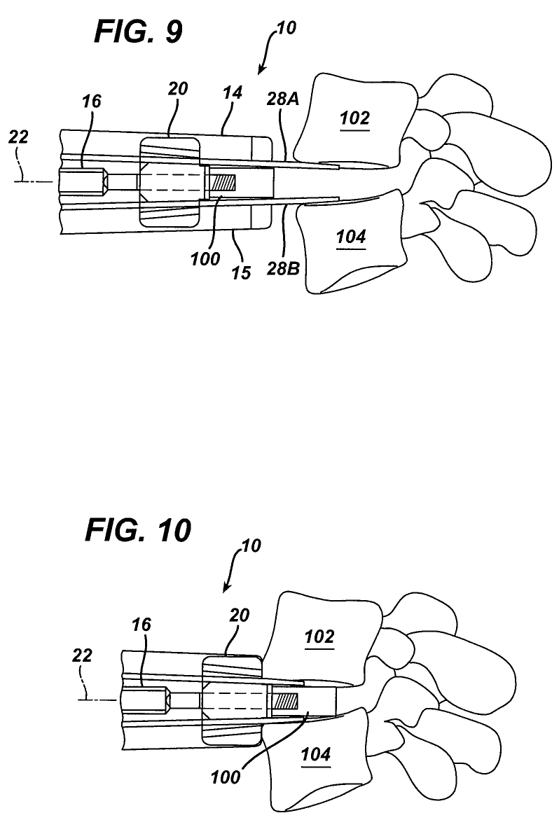

As illustrated in FIG. 9, the shaft 16 and pusher block 20 can then be advanced distally along the longitudinal axis 22 of the installation tool 10. For example, with the tool 10 in a shaft translation mode, rotation of a handle 18 (FIG. 1) of the tool 10 will cause the shaft 16 to translate along the longitudinal axis 22 and advance the pusher block 20 and prosthetic device 100 and the prosthetic device 100 toward the vertebral bodies 102, 104. As a result, the distal movement of the pusher block 20 and the prosthetic device 100 between the levers 14, 15 will cause the blade tips 28A, 28B to distract which, in turn, causes distraction of the vertebral bodies 102, 104. Advancement of the pusher block 20 continues until, as shown in FIG. 10, the prosthetic device 100 is properly installed between the adjacent vertebral bodies 102, 104. When the implant reaches its final position, continued translation of the shaft draws the opposed levers from the disc space leaving only the implant in the disc space. FIGS. 8-12 illustrate that at all times separation of the vertebral bodies is only effected to the extent necessary to insert the prosthetic device. Excessive distraction or separation of the vertebral bodies does not occur because the separation of vertebral bodies is caused by the height of the pusher block and/or the prosthetic device.

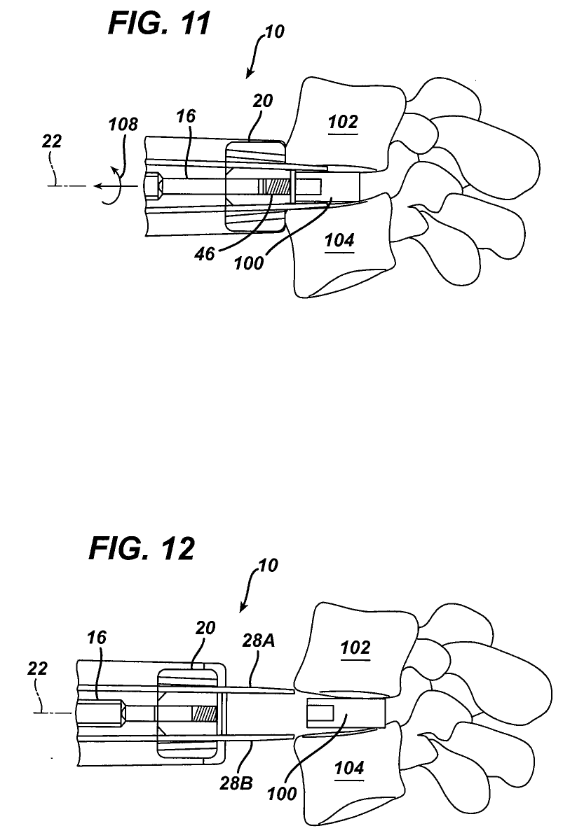

Following insertion of the prosthetic device 100, as shown in FIG. 11, if the shaft is connected directly to a prosthesis, the tool can be reconfigured in a shaft rotation mode of operation to detach the shaft 16 from the prosthetic device 100. In this manner, rotation of the handle 18 (FIG. 1) will cause the shaft 16 to rotate 108 about the longitudinal axis 22 of the installation tool 10 to decouple the threaded portion 46 of the shaft 16 from the prosthetic device 100. Once the shaft 16 has been disconnected from the prosthetic device 100, the insertion tool 10 can be removed from between the adjacent vertebral bodies 102, 104. For example, the tool can be reconfigured in a shaft translation mode of operation such that further linear translation of the shaft 16 toward the vertebral bodies 102, 104 will cause the pusher block 20 to apply a force to the vertebral bodies 102, 104 which, in turn, will cause the blade tips 28A, 28B to retract from between the vertebral bodies 102, 104 leaving only the prosthetic device 100 in the disc space.

The installation tool of the present invention can also be provided as a kit having modular components which allow the surgeon to select from among a variety of components to assemble an installation tool that is optimized for its intended use. The kit preferably includes several different shafts, pusher blocks, and other elements, each adapted to be used with a particular type or size of implant. For example, the kit can include different types of pusher blocks, each adapted to mate with a particular prosthesis. A person skilled in the art will appreciate that the installation tool can include a variety of components having a combination of different features. Moreover, the components can be adapted for use with particular types of prosthesis, or for use with other components.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A medical device installation tool, comprising:
a housing;
a pair of opposed levers, each having a proximal end and a distal end, the proximal end of each lever being moveably coupled to a portion of the housing; and
a prosthesis positioning mechanism, at least a portion of which is disposed between the pair of opposed levers, the prosthesis positioning mechanism being selectively configured such that at least a portion of the prosthesis positioning mechanism translates along a longitudinal axis of the installation tool while maintaining a substantially fixed length of the installation tool.

2. The medical device installation tool of claim 1, wherein the prosthesis positioning mechanism comprises a shaft at least partially disposed within the housing.

3. The medical device installation tool of claim 2, wherein the shaft comprises a threaded distal end adapted to couple to a prosthesis.

4. The medical device installation tool of claim 2, further comprising a driver coupled to the shaft, the driver adapted to be configured to linearly move the shaft along the longitudinal axis of the installation tool.

5. The medical device installation tool of claim 4, wherein the driver is threadably mated to the shaft.

6. The medical device installation tool of claim 5, wherein the shaft comprises a threaded proximal end and the driver includes a drive shaft having a bore with threads configured to mate with the threaded proximal end of the shaft, the driver being configurable to rotate about the longitudinal axis of the installation tool to cause translational movement of the shaft along the longitudinal axis of the installation tool.

7. The medical device installation tool of claim 2, wherein the prosthesis positioning mechanism further comprises a pusher block coupled to the shaft and disposed between the pair of opposed levers.

8. The medical device installation tool of claim 7, wherein the pusher block comprises a connection mechanism that enables the pusher block to couple directly to a prosthesis.

9. The medical device installation tool of claim 7, wherein the shaft further comprises a threaded distal end extending beyond a distal face of the pusher block and configured to couple with a prosthesis.

10. The medical device installation tool of claim 1, wherein a portion of the prosthesis positioning mechanism is further configured to selectively rotate about the longitudinal axis of the installation tool.

11. The medical device installation tool of claim 10, wherein the prosthesis positioning mechanism comprises a shaft at least partially disposed within the housing.

12. The medical device installation tool of claim 11, comprising a driver effective to selectively control the translation and the rotation of the shaft.

13. The medical device installation tool of claim 12, further comprising an actuator adapted to be configured between a first position that allows the driver to control translation of the shaft along the longitudinal axis of the installation tool and a second position that allows the driver to control rotation of the shaft about the longitudinal axis of the installation tool.

14. The medical device installation tool of claim 10, wherein the prosthesis positioning mechanism comprises a shaft at least partially disposed within the housing and having a threaded distal end adapted to be coupled to a prosthesis.

15. The medical device installation tool of claim 1, wherein the proximal end of each lever is moveably coupled to a portion of the housing.

16. The medical device installation tool of claim 1, wherein the proximal end of each lever is coupled to a portion of the housing via a coupling mechanism that allows linear translation of each lever relative to the housing.

17. A medical device installation tool, comprising:
a housing;
a shaft coupled to the housing, the shaft being selectively configured to translate along a longitudinal axis of the installation tool and to rotate about the longitudinal axis of the installation tool as a result of manipulation of a single driver; and
a pair of opposed levers, each having a proximal end and a distal end, the proximal end of each lever being pivotably coupled to a portion of the housing such that the distal ends of the levers separate in response to the shaft moving from the proximal end to the distal end.

18. The medical device installation tool of claim 17, further comprising an actuator adapted to be configured between a first position that allows the driver to control translation of the shaft along the longitudinal axis of the installation tool and a second position that allows the driver to control rotation of the shaft about the longitudinal axis of the installation tool.

19. The medical device installation tool of claim 17, wherein the shaft comprises a threaded distal end adapted to be coupled to a prosthesis.

20. The medical device installation tool of claim 17, further comprising a pusher block coupled to the shaft and disposed between the pair of opposed levers.

21. The medical device installation tool of claim 17, wherein the shaft is selectively configured to translate along a longitudinal axis of the installation tool and to rotate about the longitudinal axis of the installation tool while maintaining a substantially fixed length of the installation tool.

22. A method for implanting a prosthetic device, comprising:
disposing portions of opposed, pivotable levers of an installation tool between vertebral bodies;
linearly translating a shaft along a longitudinal axis of the installation tool to move a prosthetic device between the opposed levers toward the vertebral bodies while causing distal ends of the opposed levers to separate and distract the vertebral bodies while substantially maintaining a length of the installation tool; and
implanting the prosthetic device between the distracted vertebral bodies.

23. The method of claim 22 further comprising rotating the shaft about its longitudinal axis to decouple the shaft of the installation tool from the prosthetic device.
